(19) United States
(12) Patent Application Publication
(10) Pub. No.: US 2026/0263059 A1
(43) Pub. Date: Sep. 10, 2026

(54) SNARE WIRE STRUCTURE

(71) Applicants: TOKUSEN KOGYO CO., LTD., Hyogo (JP); KABUSHIKI KAISHA TOP, Tokyo (JP)

(72) Inventors: Naohiro Yamashita, Hyogo (JP); Hiroshi Saito, Tokyo (JP); Kouhei Goto, Tokyo (JP)

(21) Appl. No.: 19/165,409

(22) PCT Filed: May 17, 2024
(86) PCT No.: PCT/JP2024/018350

§ 371 Date: Sep. 15, 2025

(30) Foreign Application Priority Data
May 19, 2023 (JP) 2023-083504

(51) Int. Cl. A61B 17/00

(52) U.S. Cl. CPC A61B 17/00234; A61B 2017/00358

(57) ABSTRACT

A snare wire structure 1 that constitutes an endoscopic snare has an operation wire and a snare loop formed in a loop shape. The snare loop is symmetrically formed, with reference to a center line passing through a continuous portion to a tip end portion of the operation wire, by a pair of proximal end portions 120, a pair of first portions 121, and a pair of second portions. The first portion extends from an end portion of the proximal end portion to form a first designated interior angle θ₁=180°−(θ₀+δθ₁)(0.5°≤δθ₁≤12°) with respect to the proximal end portion. The second portion extends from an end portion of the first portion to form a second designated interior angle θ₂=θ₁+δθ₂(−8°≤δθ₂≤14°) with respect to the first portion. The snare loop is composed of a first strand having a twisted configuration of 1×N₁(N₁=3 to 19) and a diameter of 0.2 to 0.35 mm.

### TECHNICAL FIELD

The present invention relates to an endoscopic snare that is used in a state of being inserted into a treatment tool passage in an endoscope, and particularly, to a snare wire structure that constitutes the endoscopic snare.

### BACKGROUND ART

In the related art, an endoscopic snare has been known as an endoscope treatment tool that is inserted through a treatment tool insertion port such as a forceps port of an endoscope and is used to resect a lesion part such as a polyp. An endoscopic snare includes a snare wire structure having an operation wire inserted into an internal space of a tubular sheath and a snare loop composed of a loop-shaped or annular snare wire continuous to a tip end portion of the operation wire, and has a function of constricting (clamping) a lesion part by means of the snare loop and resecting the lesion part (for example, see Patent Literature 1).

As a treatment using the endoscopic snare, a so-called hot snare (hot polypectomy) in which a high-frequency current is allowed to flow through a snare loop to cauterize a lesion part is known. However, as the number of cases of colorectal tumors has increased, the number of endoscopic treatments has increased, and it has been desired to perform endoscopic treatment in general gastroenterology clinics. In addition, there have been increasing needs for even safer and less invasive endoscopic treatment due to the aging of patients. In order to meet these needs, a so-called cold snare (cold snare polypectomy) (hereinafter, sometimes referred to as CSP) has recently begun to spread. CSP is a technique in which a polyp is constricted with a snare and resected bluntly without a procedure of local injection and energization of the polyp.

### CITATION LIST

### Patent Literature

Patent Literature 1: Japanese Patent Application Laid-Open No. 2018-57418

### SUMMARY OF INVENTION

### Technical Problem

However, since there are few types of dedicated snares for CSP, the snare for EMR is often used, and since the dedicated snares for CSP are not energized unlike EMR and are blunt (physical) resection, the cutting ability is extremely poor, and the resection is performed by division. However, this makes it difficult to make an accurate pathological diagnosis, and the resection line may be shifted, resulting in a positive resection margin and leaving a tumor (Hirose R and Yoshida N et al. Dig Endosc 2017; 29:594-601). This is a disadvantage of the CSP in the current clinical practice.

Accordingly, an object of the present invention is to provide a snare wire structure that constitutes an endoscopic snare suitable for cold snare (cold polypectomy).

### Solution to Problem

A snare wire structure according to the present invention includes
an operation wire configured to be inserted into a through-passage of a sheath constituting an endoscopic snare, and a snare loop configured to have both ends connected to a tip end portion of the operation wire and to be formed in a loop shape, in which
the snare loop is symmetrically formed, with reference to a center line passing through a continuous portion with respect to the tip end portion of the operation wire, by
   a pair of proximal end portions that are continuous with the tip end portion of the operation wire so as to spread apart from each other at one end portion by forming a proximal end interior angle 2θ₀=55° to 80° or 60° to 80°, preferably 62° to 78°,
   a pair of first portions that form a first designated interior angle θ₁=180°−(θ₀+δθ₁)(0.5°≤δθ₁≤12° or 2°≤δθ₁≤7°) with respect to each of the pair of proximal end portions and that are continuous with the other end portion of each of the pair of proximal end portions, and
   a pair of second portions that form a second designated interior angle θ₂=θ₁+δθ₂(−8°≤δθ₂≤14° or 2°≤δθ₂≤8°) with respect to each of the pair of first portions at one end portion, that are continuous with the other end portion of each of the pair of first portions, and that are continuous at the other end portion, and
the snare loop is composed of a first strand made of stainless steel and has a twisted configuration of 1×N₁ (N₁=3 to 19 or N₁=3 to 7) and a diameter of 0.2 to 0.35 mm.

With the snare wire structure according to the configuration, due to the constriction of the lesion part by the snare loop, during the snare loop is gradually drawn into a hollow space of a sheath from the pair of proximal end portions, the snare loop is capable of being made to have appropriate resilience and hardness, and thus appropriate form-retaining property, from the viewpoint of preventing the snare loop from bouncing or slipping from a target constriction point of the lesion part. In addition, from the viewpoint of improving the gripping force by the snare loop on the lesion part covered with the mucous membrane, an appropriate uneven structure is realized on a surface of the strand of the twisted configuration 1×N₁ (N₁=3 to 19 or N₁=3 to 7). As a result, an improvement in the cutting ability of the lesion part by the snare loop achieved.

In the snare wire structure according to the above configuration,
the operation wire may be preferably composed of a second strand made of stainless steel, having a twisted configuration of 1×N₂ (N₂=3 to 7 (N₁<N₂) and having a larger diameter than the first strand.

With the snare wire structure according to the configuration, the second strand constituting the operation wire has a larger diameter than the first strand constituting the snare loop. Accordingly, the operation wire is able to have appropriate stiffness and/or strength from the viewpoint of efficiently causing the pulling force of the operation wire to act on the snare loop as a force for drawing the snare loop into the hollow space of the sheath. As a result, a further improvement in the cutting ability of the lesion part by the snare loop is achieved.

In the snare wire structure according to the above configuration,
a ratio of a length of the first portion along the center line to a length of the snare loop along the center line may be preferably in a range of 0.11 to 0.37.

With the snare wire structure according to the configuration, due to the constriction of the lesion part by the snare loop, during the snare loop is gradually drawn into the hollow space of the sheath from the pair of proximal end portions, the snare loop having an appropriate shape, particularly a length of the first portion, is realized from the viewpoint of preventing the snare loop from bouncing or slipping from a target constriction point of the lesion part. As a result, a further improvement in the cutting ability of the lesion part by the snare loop is achieved.

In the snare wire structure according to the above configuration,
the snare loop may be preferably formed by being bent or curved inward at an intermediate portion of each of the pair of second portions.

With the snare wire structure according to the configuration, the snare loop is formed by being bent or curved inward (more inward than a straight line connecting the continuous portion of each first portion and each second portion and the continuous portion of the pair of second portions) at the intermediate portion of each of the pair of second portions. That is, the snare loop is formed such that a width of the snare loop (the interval in the direction perpendicular to the center line of the snare loop) relatively rapidly decreases from the continuous portion of each first portion and each second portion to the continuous portion of the pair of second portions up to the intermediate portion, and relatively gently decreases after the intermediate portion. Therefore, in a case where the spare loop is gradually drawn into the hollow space of the sheath from the pair of proximal end portions, the gripping force of the lesion part due to the curved portion or the bending portion is improved, and the pulling force of the operation wire is capable of being efficiently converted into the constriction force of the snare loop, so that the cutting ability of the snare loop is improved.

In the snare wire structure according to the above configuration,
the snare loop may be preferably formed by being bent or curved inward at an intermediate portion of each of the pair of proximal end portions.

With the snare wire structure according to the configuration, the snare loop is formed by being bent or curved inward (more inward than a straight line connecting the continuous portion of the tip end portion of the operation wire and each proximal end portion and the continuous portion of each proximal end portion and each first portion) at the intermediate portion of each of the pair of proximal end portions. That is, the snare loop is formed such that the width of the snare loop (the interval in the direction perpendicular to the center line of the snare loop) relatively gently increases from the continuous portion of the pair of proximal end portions with respect to the tip end portion of the operation wire to the continuous portion of each of the pair of proximal end portions and each of the pair of first portions, and relatively rapidly increases from the middle. Therefore, the resistance in a case where the pair of proximal end portions, particularly, the portion where the width is gradually increased is drawn into the hollow space of the sheath is reduced. As a result, a situation in which the snare loop is slipped (bounced) from the target constriction point is avoided, and the pulling force of the operation wire is capable of being efficiently converted into the constriction force of the snare loop, so that the cutting ability of the snare loop is improved.

### BRIEF DESCRIPTION OF DRAWINGS

FIG. 1 is a plan view of a snare wire structure according to an embodiment of the present invention.

FIG. 2 is a plan view of a snare loop according to the embodiment of the present invention.

FIG. 3 is a plan view of an endoscopic snare having the snare wire structure.

FIG. 4 is a plan view of a snare loop according to another embodiment of the present invention.

### DESCRIPTION OF EMBODIMENTS

### (Configuration of Snare Wire Structure)

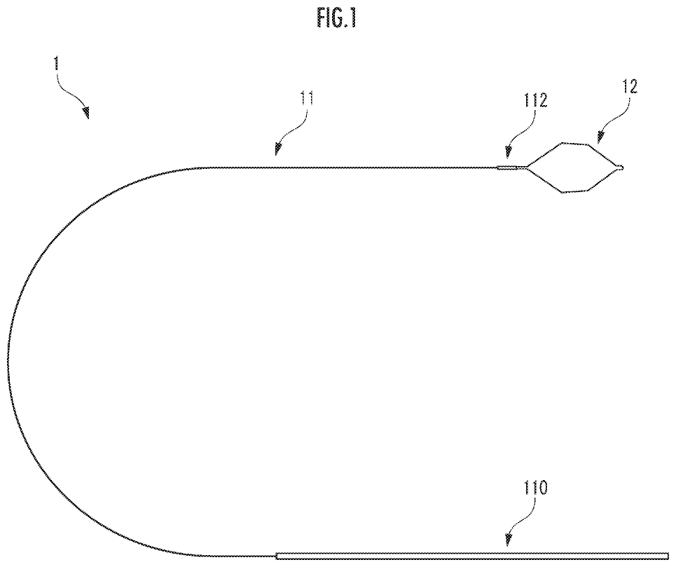

A snare wire structure 1 as an embodiment of the present invention illustrated in FIG. 1 includes an operation wire 11 and a snare loop 12 that is formed in a loop shape. The operation wire 11 is inserted into a through-passage of a sheath 20 constituting an endoscopic snare 2, which will be described later. A rear end portion 110 of the operation wire 11 is fixed to a slider 222 constituting an operating mechanism 22 of the endoscopic snare 2, which will be described later. Both ends of the snare loop 12 are connected to a tip end portion 112 of the operation wire 11.

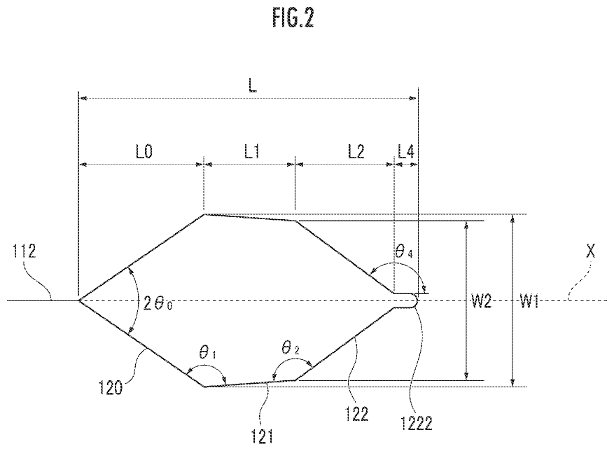

As illustrated in FIG. 2, the snare loop 12 is formed in a substantially hexagonal shape that is symmetrical with respect to a center line X (see a dashed line) passing through a continuous portion with respect to the tip end portion 112 of the operation wire 11. The spare loop 12 includes a pair of proximal end portions 120, a pair of first portions 121, and a pair of second portions 122.

Each of the pair of proximal end portions 120 is continuous with the tip end portion 112 of the operation wire 11 so as to be spread apart from each other at one end portion (left end portion in FIG. 2) by forming a proximal end interior angle 2θ₀=55° to 80° or 60° to 80°. For example, θ₀ is “34.5°”. A ratio (L₀/L) of a length L₀ of the proximal end portion 120 along the center line X to a length L of the snare loop 12 along the center line X is in a range of 0.32 to 0.45. The pair of proximal end portions 120 of the snare loop 12 (or a pair of wires extending from each proximal end of the pair of proximal end portions 120) is inserted into a tubular member made of stainless steel provided at the tip end portion 112 of the operation wire 11 together with the operation wire 11, and is connected by crimping, brazing, or welding.

Each of the pair of first portions 121 forms a first designated interior angle θ₁=180°−(θ₀+δθ₁)(0.5°≤δθ₁≤12° or 2°≤δθ₁≤7°) with respect to each of the pair of proximal end portions 120 at one end portion (left end portion in FIG. 2) and is continuous with respect to the other end portion (right end portion in FIG. 2) of each of the pair of proximal end portions 120. θ₁ is, for example, “141°” (801 is, for example, “4.5°”). A ratio (L₁/L) of a length L₁ of the first portion 121 along the center line X to the length L of the snare loop 12 along the center line X is in a range of 0.11 to 0.37 or 0.23 to 0.37. An interval W₁ (=2L₀ tan θ₀) between the continuous portions of each of the pair of proximal end portions 120 and each of the pair of first portions 121 and a ratio (W₁/L) of the interval W₁ to the length L of the snare loop 12 along the center line X are in a range of 0.45 to 0.64.

Each of the pair of second portions 122 forms a second designated interior angle θ₂=θ₁+δθ₂ (−8°≤δθ₂≤14° or 2°≤δθ₂≤8°) with respect to each of the pair of first portions 121 at one end portion (left end portion in FIG. 2) and is continuous with respect to the other end portion (right end portion in FIG. 2) of each of the pair of first portions 121. θ₂ is, for example, “147°” (δθ₂ is, for example, “6°”). A ratio (L₂/L) of a length L₂ of the second portion 122 along the center line X to the length L of the snare loop 12 along the center line X is in a range of 0.24 to 0.34. A ratio (W₂/L) of an interval W₂ (=W₁+2L₁ tan (θ₀+θ₁)) between the continuous portions of each of the pair of first portions 121 and each of the pair of second portions 122 to the length L of the snare loop 12 along the center line X is included in a range of 0.40 to 0.59.

At an intermediate portion, each of the pair of second portions 122 forms a designated external angle θ₄=θ₀+θ₁+θ₂−180°+δθ₄(0°≤δθ₄≤27° or 0°≤δθ₄≤4°) and is bent inward. θ₄ is, for example, 143° (δθ₄ is, for example, “0.5°”). A distal end portion 1222 after the intermediate portion of each of the pair of second portions 122 is continuous at the center line X, and thus extends in a substantially semi-elliptical shape or a substantially semi-ellipsoidal shape as a whole. The distal end portions 1222 of the pair of second portions 122 may be formed in various shapes, such as a substantially semicircular shape, a substantially isosceles triangular shape, or a substantially isosceles triangular shape in which the opposite sides are slightly bulged outward, as a whole.

The snare loop 12 is composed of a first strand having a twisted configuration of 1×N₁ (N₁=3 to 19 or 3 to 7) and a diameter of 0.2 to 0.35 mm. The first strand is composed of, for example, N₁ elemental wires (for example, three wires) made of stainless steel (for example, SUS304) having a diameter of 0.06 to 0.18 mm, and a diameter thereof is, for example, 0.33 mm. The operation wire 11 is composed of a second strand having a twisted configuration of 1×N₂ (N₂₌₃ to 7) and a diameter of 0.6 to 0.9 mm. The second strand is composed of, for example, N₂ elemental wires (for example, seven wires) made of stainless steel having a diameter of 0.18 to 0.45 mm, and a diameter thereof is, for example, 0.80 mm. The diameter of the operation wire 11 is configured to be slightly smaller than an inner diameter of the sheath 20 into which the operation wire 11 is inserted.

### (Configuration of Endoscopic Snare)

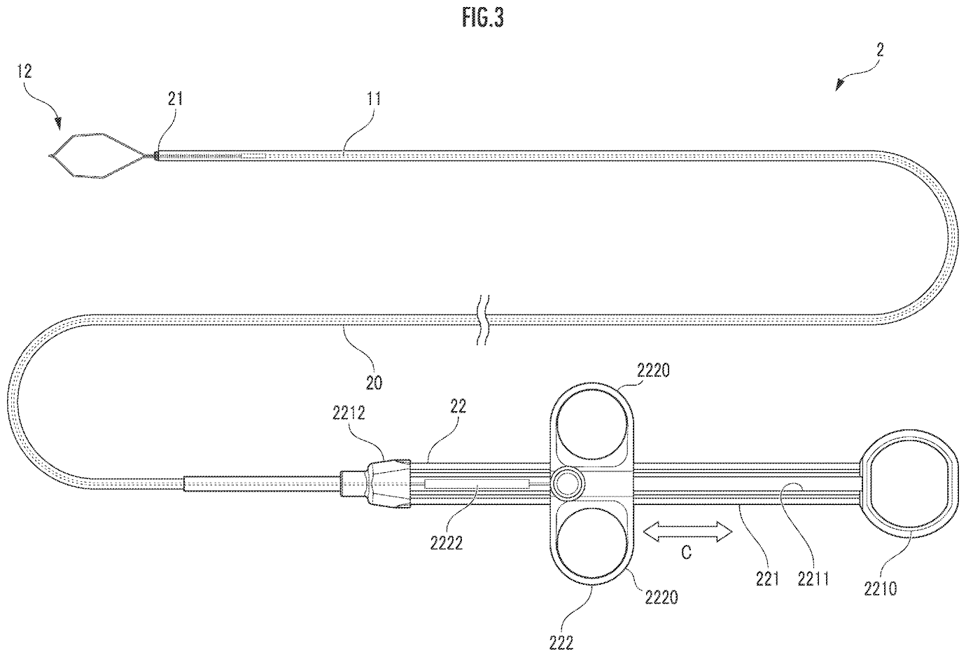

The snare wire structure 1 illustrated in FIG. 1 constitutes the endoscopic snare 2 illustrated in FIG. 3. As illustrated in FIG. 3, the endoscopic snare 2 includes the sheath 20 and the operating mechanism 22. The endoscopic snare 2 is inserted from a treatment tool channel opening portion, such as a forceps port, of the endoscope and is inserted into the treatment tool channel.

The sheath 20 is a flexible tubular member, and has sliding properties (low friction properties) of an inner surface allowing the sheath 20 to be flexibly bent together with the endoscope and allowing the operation wire 11 passed through the hollow space to smoothly move forward and backward. The sheath 20 is formed of, for example, polytetrafluoroethylene (PTFE), which is a type of fluororesin having flexibility and sliding properties, high-density polyethylene (HDPE) having higher hardness than PTFE, fluororesins such as PTFA, ETFE, FEP, PVDF, PCFFE, or ECTFE, or other synthetic resins.

The operating mechanism 22 includes a base 221 to which the sheath 20 is connected, and a slider 222 to which the operation wire 11 is connected. The base 221 and the slider 222 are formed of a synthetic resin having sufficient strength, such as polycarbonate (PC)

The base 221 is formed to extend in a longitudinal direction of the base 221 and has a base groove 2211 for accommodating the operation wire 11. By forming the base groove 2211, a cross-sectional shape of the base 221 is substantially U-shaped. The slider 222 is loosely fitted to an outer periphery of the base 221 and is slidably supported in the longitudinal direction or in a front-back direction of the base 221. A rear end portion of the sheath 20 is connected to a tip end portion of the base 221, and a rear end portion 110 of the operation wire 11 is connected to the slider 222. Therefore, the slider 222 is relatively moved with respect to the base 221, so that the operation wire 11 is relatively driven forward and backward with respect to the sheath 20, and it is possible to change the protruding amount of the snare loop 12 from the sheath 20.

In addition, a substantially cylindrical stopper member 2222 made of PTFE is disposed between the slider 222 and a cap 2212 in the base groove 2211, and the rear end portion 110 of the operation wire 11 is inserted into and fixed to a hollow space of the stopper member 2222. The stopper member 2222 is pushed by the slider 222 and abuts on the cap 2212, whereby a position on the most tip end side of the movable range of the slider 222 is set, and thus the maximum protruding amount of the snare loop 12 is set.

A first operation ring 2210 into which a user inserts a thumb is provided at an end portion of the base 221 on a side opposite to the snare loop. In addition, two second operation rings 2220 into which, for example, a user's index finger and middle finger are inserted are provided on both sides of the slider 222 in a direction orthogonal to the longitudinal direction of the base 221. Accordingly, the operating mechanism 22 is capable of being operated with one hand. In a case where the snare loop 12 is accommodated in the sheath 20, the slider 222 abuts the first operation ring 2210, thereby determining the limit of the snare loop 12 being drawn into the sheath 20 and also functioning as a retaining member for the slider 222.

### (Action and Effect of Snare Wire Structure)

The snare loop 12 is composed of a first strand made of stainless steel and having a twisted configuration of 1×N₁ (N₁=3 to 19 or 3 to 7) and a diameter of 0.2 to 0.35 mm. Accordingly, during the snare loop 12 is gradually drawn into the hollow space of the sheath 20 from the pair of proximal end portions 120, the snare loop 12 is capable of being made to have appropriate resilience and hardness, and thus appropriate form-retaining property, from the viewpoint of preventing the snare loop 12 from bouncing or slipping from a target constriction point of the lesion part. In addition, from the viewpoint of improving the gripping force by the snare loop 12 on the lesion part covered with the mucous membrane, an appropriate uneven structure is realized on a surface of the strand of the twisted configuration 1×N₁ (N₁=3 to 19 or 3 to 7). As a result, an improvement in the cutting ability of the lesion part by the snare loop 12 is achieved.

The operation wire 11 is composed of a second strand made of stainless steel, which has a twisted configuration of 1×N₂ (N₂=3 to 7 (N₁<N₂)) and a larger diameter than the first strand. Accordingly, the operation wire 11 is able to have appropriate stiffness and/or strength from the viewpoint of efficiently converting the pulling force of the operation wire 11 into the constriction force of the snare loop 12 in a case of being drawn into the hollow space of the sheath 20. As a result, a further improvement in the cutting ability of the lesion part by the snare loop 12 is achieved.

The ratio (L₁/L) of the length L₁ of the first portion 121 along the center line X to the length L of the snare loop 12 along the center line X is in a range of 0.11 to 0.37 or 0.23 to 0.37. Therefore, during the snare loop 12 is gradually drawn into the hollow space of the sheath 20 from the pair of proximal end portions 120, the snare loop 12 having an appropriate shape, particularly a length of the first portion 121, is realized from the viewpoint of preventing the snare loop 12 from bouncing or slipping from a target constriction point of the lesion part. As a result, a further improvement in the cutting ability of the lesion part by the snare loop 12 is achieved.

The snare loop 12 is formed to be bent or curved inward at an intermediate portion of each of the pair of second portions 122 (see FIG. 2). That is, the snare loop 12 is formed such that a width of the snare loop 12 (the interval in the direction perpendicular to the center line X of the snare loop 12) relatively rapidly decreases from the continuous portion of each first portion 121 and each second portion 122 to the continuous portion of the pair of second portions 122 and relatively gently decreases in the distal end portion 1222 after the intermediate portion. Therefore, in a case where the snare loop 12 is gradually drawn into the hollow space of the sheath 20 from the pair of proximal end portions 120, the gripping force of the lesion part due to the curved portion or the bending portion is improved, and the pulling force of the operation wire 11 is capable of being efficiently converted into the constriction force of the snare loop 12, so that the cutting ability of the snare loop 12 is improved.

### Other Embodiments of Present Invention

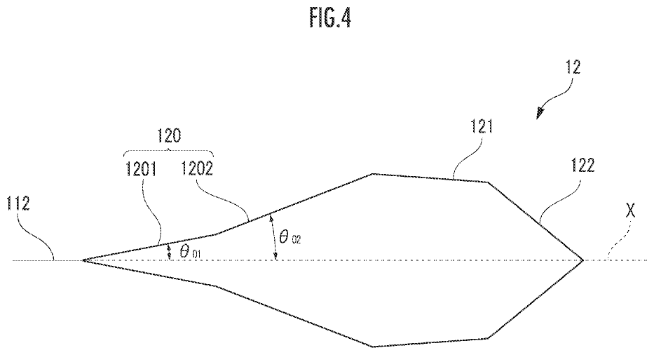

In the above embodiment, the snare loop 12 is formed such that each of the pair of second portions 122 is bent (or curved) inward at the intermediate portion to form the designated external angle θ₄ (more inward than a straight line connecting the continuous portion of each of the pair of first portions 121 and each of the pair of second portions 122 and the continuous portion of each of the pair of second portions 122 and the distal end portion 1222). However, as another embodiment, as illustrated in FIG. 4, each of the pair of second portions 122 may be formed so as to generally extend along the straight line.

As illustrated in FIG. 4, the snare loop 12 may be formed so as to be bent (or curved) inward at the intermediate portion of each of the pair of proximal end portions 120. For example, as illustrated in FIG. 4, the proximal end portion 120 may be configured with a first proximal end portion 1201 and a second proximal end portion 1202 that is continuous with an end portion of the first proximal end portion 1201 so as to be bent to form an obtuse external angle. The first proximal end portion 1201 is continuous with the tip end portion 112 of the operation wire 11, with the continuous portion therebetween serving as a center, and forms an angle θ₀₁ with respect to the center line X. The second proximal end portion 1202 is continuous with the tip end portion 112 of the operation wire 11, with the continuous portion therebetween serving as a center, and forms an angle θ₀₂ (θ₀₁<θ₀₂) with respect to the center line X.

With the snare wire structure 1 according to the configuration, the snare loop 12 is formed by being bent inward (more inward than a straight line connecting the continuous portion of the tip end portion 112 of the operation wire 11 and each proximal end portion 120 and the continuous portion of each proximal end portion 120 and each first portion 121) at the intermediate portion of each of the pair of proximal end portions 120. That is, the snare loop 12 is formed such that the width of the snare loop in the first proximal end portion 1201 (the interval in the direction perpendicular to the center line of the snare loop) relatively gently increases from the continuous portion of the pair of proximal end portions 120 with respect to the tip end portion 112 of the operation wire 11 to the continuous portion of each of the pair of proximal end portions 120 and each of the pair of first portions 121, and relatively rapidly increases in the second proximal end portion 1202 from the middle. Therefore, the resistance in a case where the pair of proximal end portions 120, particularly, the first proximal end portion 1201 where the width is gradually increased is drawn into the hollow space of the sheath 20 is reduced. As a result, a situation in which the snare loop 12 is slipped (bounced) from the target constriction point of the lesion part is avoided, and the pulling force of the operation wire 11 is capable of being efficiently converted into the constriction force of the snare loop 12, so that the cutting ability of the snare loop 12 is improved.

### DESCRIPTION OF REFERENCE NUMERALS

1: snare wire structure
11: operation wire
12: snare loop
120: proximal end portion
121: first portion
122: second portion
2: endoscopic snare
20: sheath
22: operating mechanism

1-5. (canceled)

6. A snare wire structure, comprising:
an operation wire configured to be inserted into a through-passage of a sheath constituting an endoscopic snare; and
a snare loop configured to have both ends connected to a tip end portion of the operation wire and to be formed in a loop shape,
wherein the snare loop is symmetrically formed, with reference to a center line passing through a continuous portion with respect to the tip end portion of the operation wire, by
a pair of proximal end portions that are continuous with the tip end portion of the operation wire so as to spread apart from each other at one end portion by forming a proximal end interior angle 2θ₀=55° to 80°,
a pair of first portions that form a first designated interior angle θ₁=180°−(θ₀+δθ₁)(0.5°≤δθ₁≤12°) with respect to each of the pair of proximal end portions and that are continuous with an other end portion of each of the pair of proximal end portions, and
a pair of second portions that form a second designated interior angle θ₂=θ₁+δθ₂ (−8°≤δθ₂≤14°) with respect to each of the pair of first portions at one end portion, that are continuous with an other end portion of each of the pair of first portions, and that are continuous at an other end portion, and
the snare loop includes a first strand made of stainless steel and has a twisted configuration of 1×N₁ (N₁=3 to 19) and a diameter of 0.2 to 0.35 mm.

7. The snare wire structure according to claim 6,
wherein the operation wire includes a second strand made of stainless steel, having a twisted configuration of 1×N₂ (N₂=3 to 7 (N₁<N₂)) and having a larger diameter than the first strand.

8. The snare wire structure according to claim 6,
wherein a ratio of a length of the first portion along the center line to a length of the snare loop along the center line is in a range of 0.11 to 0.37.

9. The snare wire structure according to claim 6,
wherein the snare loop is formed by being bent or curved inward at an intermediate portion of each of the pair of second portions.

10. The snare wire structure according to claim 6,
wherein the snare loop is formed by being bent or curved inward at an intermediate portion of each of the pair of proximal end portions.

11. The snare wire structure according to claim 7,
wherein a ratio of a length of the first portion along the center line to a length of the snare loop along the center line is in a range of 0.11 to 0.37.

12. The snare wire structure according to claim 7,
wherein the snare loop is formed by being bent or curved inward at an intermediate portion of each of the pair of second portions.

13. The snare wire structure according to claim 7,
wherein the snare loop is formed by being bent or curved inward at an intermediate portion of each of the pair of proximal end portions.
